(19) United States
(12) Patent Application Publication
(10) Pub. No.: US 2024/0366704 A1
(43) Pub. Date: Nov. 7, 2024

(54) GROW FORMULA

(71) Applicant: Andrea Murillo Dinh, Irvine, CA (US)

(72) Inventor: Andrea Murillo Dinh, Irvine, CA (US)

(21) Appl. No.: 18/655,183

(22) Filed: May 3, 2024

Related U.S. Application Data
(60) Provisional application No. 63/500,245, filed on May 4, 2023.

(51) Int. Cl. A61K 36/42; A61K 31/198

(52) U.S. Cl. CPC A61K 36/42; A61K 31/198

(57) ABSTRACT

Provided herein are formula compositions for promoting growth and treatment of moderate acute malnutrition or severe acute malnutrition by increasing lean muscle mass in a patient in need thereof, the formula composition having: at least one animal-based amino acid; at least one vegetable-based amino acid; and Cucurbita maxima; wherein the patient is approximately 2 years old to approximately 6 years old.

### CROSS-REFERENCE TO RELATED APPLICATIONS

This application claims the benefit of U.S. Provisional Application No. 63/500,245, filed May 4, 2023, which is hereby incorporated by reference, to the extent that it is not conflicting with the present application.

### BACKGROUND OF INVENTION

### 1. Field of the Invention

The invention relates generally to nutrition supplements and more particularly to supplements and grow formulas for prevention of malnutrition.

### 2. Description of the Related Art

Around 45% of deaths among children under 5 years of age can be linked to undernutrition or malnutrition. Such deaths generally occur in low- and middle-income areas where food availability is scarce. Malnutrition can occur in low-income areas such as third world countries, but can also be present in high-income countries such as the United States due to the low nutrition densities of foods consumed by some children. For example, some children that are “picky eaters” may not consume foods with high nutrition density. Other factors such as income, location, and so on may affect the nutrition densities of foods consumed by some children. In such areas, rates of childhood obesity and other conditions relating to overweightness are rising. These health issues can be attributed or linked to inadequate nutrition, which can cause other underlying health issues, such as, but not limited to, issues relating to cardiovascular health, cognitive decline, organ malfunction, and so on. Such nutrient deficiency can also be linked to economical issues in such areas.

Some existing sources of providing nutrition include ready-to-use therapeutic foods (RUTF), but these can increase weight in slight increments, and children can relapse into unhealthy weight ranges when they discontinue use of the product. Some children can go months without progressing into healthy weight ranges when discontinuing use of such products. In some cases, RUTF type foods provide high-calorie accessible meals, with some micronutrients, in order to return users to a normal or healthy weight. However, such foods often do not address the consequences of malnutrition that the user may already be suffering from due to MAM or SAM, such as, for example, gut microbiome imbalances, obesity predisposition, insulin spikes, high sodium/iron which then causes difficulties in balancing electrolytes, and iron deficiency or excess.

Some existing formulas for treating malnutrition, such as RUTF or corn/soy-based solutions, focus primarily on high carbohydrate compositions, generally coming from refined sugars, peanuts, and oils. Foods having such compositions can have problems such as promoting inflammation, and thus disrupting the healing process.

Therefore, there is a need for a solution to these problems.

The aspects or the problems and the associated solutions presented in this section could be or could have been pursued; they are not necessarily approaches that have been previously conceived or pursued. Therefore, unless otherwise indicated, it should not be assumed that any of the approaches presented in this section qualify as prior art merely by virtue of their presence in this section of the application.

### BRIEF INVENTION SUMMARY

This Summary is provided to introduce a selection of concepts in a simplified form that are further described below in the Detailed Description.

In some embodiments, provided herein is a grow formula that targets nutrient deficiencies to prevent, treat, and reverse moderate acute malnutrition (MAM) and severe acute malnutrition (SAM), provided in a sustainable way to be utilized long term, in order to save lives and improve the quality of life particularly in children approximately 2 years to approximately 6 years old.

In some embodiments, provided herein is a grow formula having a composition with a high nutrient density, having a combination of amino acids from both plant and animal sources and other added specific nutrients. In such combinations, the grow formulas provided herein can help to raise the weight of children taking the grow formula, by increasing lean muscle mass.

The above aspects or examples and advantages, as well as other aspects or examples and advantages, will become apparent from the ensuing description and accompanying drawings.

### BRIEF DESCRIPTION OF THE DRAWINGS

For exemplification purposes, and not for limitation purposes, aspects, embodiments or examples of the invention are illustrated in the figures of the accompanying drawings, in which:

FIG. 1 is a table showing an exemplary list of amino acids that can be used in any of the grow formulas disclosed herein, according to an aspect.

FIG. 2 is a table showing exemplary nutrient facts for one serving of any of the grow formulas disclosed herein, according to an aspect.

FIG. 3 is a table showing exemplary ingredients and amounts for one serving of any of the grow formulas disclosed herein, wherein the serving size is a 43 gram (g) serving, according to an aspect.

### DETAILED DESCRIPTION

What follows is a description of various aspects, embodiments and/or examples in which the invention may be practiced. Reference will be made to the attached drawings, and the information included in the drawings is part of this detailed description. The aspects, embodiments and/or examples described herein are presented for exemplification purposes, and not for limitation purposes. It should be understood that structural and/or logical modifications could be made by someone of ordinary skills in the art without departing from the scope of the invention.

It should be understood that, for clarity of the drawings and of the specification, some or all details about some structural components or steps that are known in the art are not shown or described if they are not necessary for the invention to be understood by one of ordinary skills in the art.

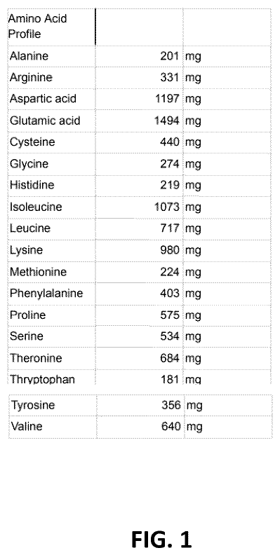

FIG. 1 is a table showing an exemplary list of amino acids that can be used in any of the grow formulas disclosed herein, according to an aspect. Provided herein are grow formulas having a unique combination of animal-based and vegetable-based amino acids, and micronutrients having both food-grade and pharmaceutical-grade sources. The amino acid profile shown in the table of FIG. 1 is an exemplary formulation of a combination of amino acids and exemplary amounts in milligrams for each of the listed amino acids. The amino acids in an exemplary composition for any of the grow formulas disclosed herein include, but are not limited to, alanine, arginine, aspartic acid, glutamic acid, cysteine, glycine, histidine, isoleucine, leucine, lysine, methionine, phenylalanine, proline, serine, threonine, tryptophan, tyrosine, and valine.

In some embodiments, the grow formulas disclosed herein are provided with a unique amino acid profile, such as the profile shown as an example in FIG. 1, combining animal- and plant-based proteins. In such embodiments, the combination of animal- and plant-based proteins enhances the absorption and synergistic effect of the provided amino acids, making the amino acids of the grow formula highly bioavailable.

In some embodiments, the grow formulas disclosed herein comprise prebiotics in a plant-based formula. In such embodies, the prebiotics can help to improve gut health in a user by feeding beneficial bacteria to the intestines when the user consumes the grow formula. This can help lead to better digestion and better absorption of nutrients.

In some embodiments, the grow formulas disclosed herein comprise a high content of omega-3 fatty acids (DHA, EPA, and ALA). Such fatty acids are associated with anti-inflammatory properties, as well as improving cognitive function, which can be vital for children suffering from malnutrition. For example, research has shown that DHA can pass through the blood-brain barrier, which is a selectively permeable membrane, via specialized transport systems involving protein 2A (MFSD2A), thus allowing nutrients into the brain.

In some embodiments, the grow formulas disclosed herein can help to improve attention span and cognitive function in users taking the grow formula. In such embodiments, the grow formulas comprise a combination of tyrosine, zinc, and magnesium found in the preparation of a pumpkin seed protein to help support cognitive function. In such embodiments, cognitive function in the user can be supported by improving transport of tyrosine into the brain. Additionally, the combination of beta-glucans and glutamate provided in the grow formulas disclosed herein can improve energy metabolism in the user by enhancing the transport of glutamate into the body's cells and thus working synergistically to enhance mitochondrial function and energy metabolism by activating AMP-activated protein kinase (AMPK) and stimulating the uptake of glucose and fatty acids into the body's cells.

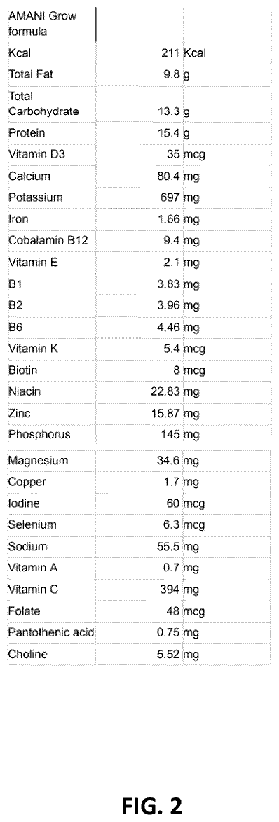

FIG. 2 is a table showing exemplary nutrient facts for one serving of 43 g any of the grow formulas disclosed herein, according to an aspect. Exemplary kcal per serving is shown as 211 kcal. Exemplary total fat per serving is shown as 9.8 grams. Exemplary total carbohydrates is shown as 13.3 grams. Exemplary total protein per serving is shown as 15.4 grams. Exemplary amounts per serving of vitamin D3, calcium, potassium, iron, cobalamin B12, vitamin E, B1, B2, B6, vitamin K, biotin, niacin, zinc, phosphorus, magnesium, copper, iodine, selenium, vitamin A, vitamin C, folate, pantothenic acid, and choline are shown in mcg or mg.

In some embodiments, the grow formulas provided herein comprise components not provided in existing grow formulas, such as any of the components listed in the table shown in FIG. 2. For example, some existing formulas do not comprise choline, selenium, and chromium. In other examples, existing formulas may be deficient in choline, selenium, and chromium.

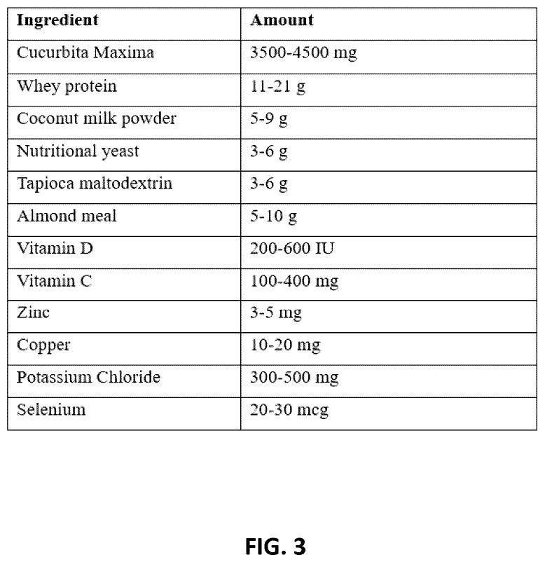

FIG. 3 is a table showing exemplary ingredients and amounts for one serving of any of the grow formulas disclosed herein, wherein the serving size is a 43 gram (g) serving, according to an aspect. It should be understood that any of the grow formulas provided herein may comprise ingredients in the amounts shown in FIG. 3, or may vary from the ingredients and the amounts shown.

In some embodiments, the grow formulas provided herein comprise Cucurbita maxima. The inclusion of Cucurbita maxima can provide a source of antioxidants and anti-inflammatory compounds that may support immune function and cardiovascular health. Additionally, roasting or baking Cucurbita maxima at high temperatures may help to release some of the bitter compounds and potentially increase its antiparasitic effects in conjunction with other ingredients of the grow formula, such as, for example, medium-chain triglycerides (MCT) and linoleic acid provided by coconut.

In some embodiments, the grow formulas provided herein comprise coconut milk powder. The inclusion of coconut milk powder can provide healthy fats and MCTs, which may support brain development and healthy weight gain in infants. Again as discussed above, the fatty acids from the coconut may help to enhance the absorption and bioavailability of the bitter compounds in Cucurbita maxima, potentially increasing their antiparasitic effects.

In some embodiments, the grow formulas provided herein comprise whey protein. The whey protein may provide a source of high-quality protein that may support muscle growth and repair. By combining animal-based and plant-based protein sources, high bioavailability can be ensured for the user taking the grow formula.

In some embodiments, the grow formulas provided herein comprise nutritional yeast. In such embodiments, the nutritional yeast can act as a catalyst to break down hydrogen peroxide (H₂O₂). High levels of H₂O₂ have shown to be detrimental in muscle growth; hence, lowering concentrations of reactive oxygen species (ROS) could have potential benefits in anabolic metabolism. In some embodiments, the grow formulas provided herein comprise tapioca maltodextrin. This ingredient is a source of carbohydrates, which provide energy for growth and development. Tapioca maltodextrin has a high glycemic index, which means it can be quickly digested and absorbed, providing a rapid source of energy.

In some embodiments, the grow formulas provided herein comprise almond meal. The almond meal provided in the grow formulas may be previously prepared to optimize amino acid functionality, which is discussed in further detail herein. The almond meal may provide a source of protein and fiber that may help support muscle growth, digestive health, and satiety.

In some embodiments, the grow formulas provided herein comprise micronutrients such as choline, vitamin D, vitamin C, zinc, and copper. These nutrients can play important roles in various aspects of infant growth and development, such as bone health, immune function, and cognitive development.

In some embodiments, the grow formulas provided herein are taken by a user by mixing the grow formula with water. In some embodiments, the grow formulas provided herein are taken by a user by adding the grow formula to food, such as a soup or porridge-based meal.

In some embodiments, the grow formulas provided herein are provided with a measuring unit for users to obtain individual serving sizes. In some embodiments, the grow formulas provided herein are provided in individual serving units.

### EXAMPLES

### Example 1: Preparation of Grow Formula

Provided herein is an exemplary method for preparation of any of the grow formulas disclosed herein.

Cooking Cucurbita maxima: An oven is preheated to approximately 180-200° C. (approximately 356-392° F.). Cucurbita maxima is cut into small cubes and placed on a baking tray lined with parchment paper, and baked in the preheated oven for approximately 30 minutes. The Cucurbita maxima is allowed to cool to room temperature before adding it to the other ingredients.

Soak and sprout almonds: Almonds are soaked and sprouted, which can help to reduce the levels of anti-nutrients, such as phytic acid, that could bind to minerals and hinder their absorption. The soaking and sprouting process can also help to improve the bioavailability of the protein and other nutrients provided by the almonds. The process is carried out by soaking the almonds in water for approximately 8-12 hours, draining and rinsing the almonds, and leaving them in a sprouting jar or container for approximately 1-2 until sprouts form.

Toasting almonds: The almonds are next toasted at a low temperature to enhance their flavor and promote the Maillard reaction, which can create new flavor compounds and potentially enhance the bioavailability of proteins provided by the almonds. The process is carried out by spreading the almonds out on a baking sheet and toasting them in a preheated oven at approximately 275° F. (approximately 135° C.) for approximately 10-15 minutes, stirring occasionally.

Grinding almonds: After the toasting process, the almonds are dried completely. Next, the almonds are ground in a food processor or high-speed blender until a fine powder or meal is formed. It should be ensured that the almonds are not overprocessed during this step to avoid the almonds release their oils and becoming sticky.

Mixing: A mixer, blender, or any other suitable device for powder blending is used for combining the ingredients of the grow formula. As an example, a speed of approximately 500-800 RPM is used for blending. The following ingredients, in amounts provided in FIG. 3 as an example, are slowly added to be mixed together: whey protein, almond meal, coconut milk powder, nutritional yeast, tapioca maltodextrin, and micronutrient mix. In some embodiments, the micronutrient mixture comprises choline, zinc, copper, selenium, vitamin C, and vitamin D. The mixture is blended until the powders are evenly distributed and a homogenous mixture is achieved. About 10-15 minutes of mixing may be necessary for some powder mixtures. The mixer is then turned off and the sides of the mixing container are scraped down to ensure all of the powder has been blended.

Storage: The blended protein powder is then transferred to an airtight container for storage.

It may be advantageous to set forth definitions of certain words and phrases used in this patent document. The term “couple” and its derivatives refer to any direct or indirect communication between two or more elements, whether or not those elements are in physical contact with one another. The term “or” is inclusive, meaning and/or. As used in this application, “and/or” means that the listed items are alternatives, but the alternatives also include any combination of the listed items.

The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

Further, as used in this application, “plurality” means two or more. A “set” of items may include one or more of such items. The terms “comprising,” “including,” “carrying,” “having,” “containing,” “involving,” and the like are to be understood to be open-ended, i.e., to mean including but not limited to. Only the transitional phrases “consisting of” and “consisting essentially of,” respectively, are closed or semi-closed transitional phrases.

Throughout this description, the aspects, embodiments or examples shown should be considered as exemplars, rather than limitations on the apparatus or procedures disclosed. Although some of the examples may involve specific combinations of method acts or system elements, it should be understood that those acts and those elements may be combined in other ways to accomplish the same objectives.

Acts, elements and features discussed only in connection with one aspect, embodiment or example are not intended to be excluded from a similar role(s) in other aspects, embodiments or examples.

Aspects, embodiments or examples of the invention may be described as processes, which are usually depicted using a flowchart, a flow diagram, a structure diagram, or a block diagram. Although a flowchart may depict the operations as a sequential process, many of the operations can be performed in parallel or concurrently. In addition, the order of the operations may be re-arranged. With regard to flowcharts, it should be understood that additional and fewer steps may be taken, and the steps as shown may be combined or further refined to achieve the described methods.

Although aspects, embodiments and/or examples have been illustrated and described herein, someone of ordinary skills in the art will easily detect alternate of the same and/or equivalent variations, which may be capable of achieving the same results, and which may be substituted for the aspects, embodiments and/or examples illustrated and described herein, without departing from the scope of the invention. Therefore, the scope of this application is intended to cover such alternate aspects, embodiments and/or examples.

What is claimed is:

1. A formula composition for promoting growth and treatment of moderate acute malnutrition or severe acute malnutrition by increasing lean muscle mass in a patient in need thereof, the formula composition comprising:
at least one animal-based amino acid;
at least one vegetable-based amino acid; and
Cucurbita maxima;
wherein the patient is approximately 2 years old to approximately 6 years old.
